(19) United States
(12) Patent Application Publication
(10) Pub. No.: US 2008/0221390 A1
(43) Pub. Date: Sep. 11, 2008

(54) Medico-Technical Device Comprising a Self-Lubricating Element

(75) Inventor: Konstantin Bob, Weinheim (DE)

Correspondence Address: STANDLEY LAW GROUP LLP, 495 METRO PLACE SOUTH, SUITE 210, DUBLIN, OH, 43017, US

(73) Assignee: INVENDO MEDICAL GMBH, Weinheim (DE)

(21) Appl. No.: 12/089,861

(22) PCT Filed: Oct. 11, 2006
(86) PCT No.: PCT/EP2006/067258

§ 371 Date: May 6, 2008

(30) Foreign Application Priority Data
Oct. 11, 2005 (DE) 10 2005 048 675.4

(51) Int. Cl. A61B 1/01

(52) U.S. Cl. 600/114

(57) ABSTRACT

The invention describes a medico-technical device adapted to be engaged with a human or animal body and comprising a self-lubricating element exposed to internal friction in the device.

### FIELD OF THE INVENTION

The present invention relates to a medico-technical device adapted to be engaged with a human or animal body.

### BACKGROUND OF THE INVENTION

Medico-technical devices which are adapted to be engaged with a human or animal body are manifold. Examples of such devices include, for instance, endoscopes, catheters, needles, etc.

Endoscopic devices as well as devices for inserting a medical endoscope into a body canal are described, for instance, in the laid-open German Patent Application DE-A-39 25 484. The devices described there permit an endoscope to be no longer pushed into the body to be examined, but to move in on its own. For this purpose, the endoscope is equipped with a self-propulsion that permits a less complicated and quicker insertion.

As a self-propulsion of this type also a so-called everting tube can be used, for instance, into which the endoscope shaft is inserted. Upon propulsion of the endoscope different relative movements occur. On the one hand, a relative movement occurs between the endoscope shaft and the everting tube which are in mutual gliding contact. On the other hand, a relative movement also occurs between an inside section and an outside section of the unwinding everting tube.

In order to reduce the respective gliding friction occurring, for example the use of a lubricant supplied from outside has been suggested, e.g. in EP-A-0 873 761. FIGS. 1 and 2 show examples of the endoscopic devices described in EP-A-0 873 761. In both cases, a lubricant is supplied between the endoscope shaft 1 and the everting tube 2, on the one hand, and between the two superimposed everting tube sections, on the other hand. This lubricant is stored in external reservoirs 3 and 4 and is pumped in during use.

### SUMMARY OF THE INVENTION

It is an object of the present invention to provide an improved medico-technical device, especially an improved endoscopic device. Specific improvements which are preferably to be achieved, are in particular a simplified structure of the medico-technical device, increased economic efficiency, improved acceptance by the individual with whom the device is used, as well as a reduction of the frictional forces occurring in the medico-technical device.

According to the invention, the object is achieved by a medico-technical device adapted to be engaged with a human or animal body comprising a self-lubricating element exposed to internal friction in the device.

In a preferred embodiment it is the self-lubricating element itself which is adapted to be engaged with a human or animal body and gets into contact with the body.

It is further preferred that at least one surface of the self-lubricating element is plasma-treated, wherein more preferably the plasma-treated surface is plasma-coated.

Advantageously the plasma-coated surface is coated with siloxane, fluorocarbon, PTFE, ePTFE and/or polyurethane.

At least one surface of the self-lubricating element is preferably surface-treated, for instance by ultraviolet radiation or silicating.

The self-lubricating element of the device according to the invention preferably may exhibit a ceramic coating, an acrylate coating and/or an aryl coating.

Moreover, it is advantageous and preferred that the self-lubricating element comprises silicone rubber, further preferred that it consists of silicon rubber.

Furthermore, the self-lubricating element may have a surface layer which comprises a material selected from the group consisting of graphite, nitride, fluorocarbon, MoS₂, WS₂, talcum, silicone oil, polysilazane, polysiloxane, PTFE, ePTFE, gelatine, agar-agar and cellulose, a combination of a polysaccharide and a protein or a silicone oil, e.g. a hydrophilic silicone oil or a hydrophobic silicone oil.

In a preferred configuration of this embodiment the surface of the self-lubricating element is hydrophilic; in another preferred configuration it is hydrophobic. In respect to water, hydrophilic surfaces have contact angles which are smaller than 90°; hydrophobic surfaces have contact angles of 90° or more, on the other hand. By contact angle the angle is meant which is formed by a tangent to the drop contour in the three-phase-point (solid-liquid-gaseous) with respect to the surface of the solid (the surface) and thus exhibits a measure for the wettability of a surface or interface by a different phase. The larger the contact angle, the lower the wetting.

In another embodiment of the invention at least one surface of the self-lubricating element shows cavities containing lubricant. It is preferred in this context that the profile can be produced by laser engraving.

It is preferred when a plasma-coating is formed over the cavities.

Furthermore, the cavities are preferably adapted in such manner that released lubricant is supplied to areas of the self-lubricating element that are exposed to higher friction than other areas.

It is especially preferred that the cavities are adapted such that, upon superposition of two opposed sections of the self-lubricating element, the respective cavity sections cannot engage.

In another embodiment of the invention the self-lubricating element has a layered structure comprising at least one substrate layer, one reservoir layer for lubricants disposed on one or both sides of the substrate and one or two external perforated layers.

Concerning the substrate layer, it is preferred that it is substantially impermeable for the lubricant. Concerning the reservoir layer, it is preferred that it is formed such that the lubricant is provided in solid form as lubricant layer. In addition, the reservoir layer may preferably include a material having a capillary effect.

In another embodiment of the invention the self-lubricating element consists of a material in which a lubricant is spread in an almost homogenous manner. It is further preferred in this context that there is an incompatibility between the lubricant and the material so that a progressive separation of the lubricant and the material takes place.

Moreover it is preferred that the self-lubricating element is porous.

Concerning the lubricant it is preferred and advantageous when the temperature-viscosity characteristic of the lubricant exhibits a discontinuity in the temperature range of from 10 to 43° C.

Apart from that, the present invention provides a medico-technical device according to any one of the embodiments described in the foregoing and hereinafter in which the self-lubricating element is tube-shaped.

Furthermore, the present invention provides an endoscopic apparatus including the device according to any one of the embodiments described above and hereinafter in which the self-lubricating element is in the form of an everting tube.

Moreover, the present invention provides the use of the device according to any one of the embodiments described before and hereinafter as everting tube and/or endoscope shaft cover (or endoscope shaft) in endoscopy.

According to the invention, moreover a method of manufacturing a medico-technical device is provided which is adapted to engage with a human or animal body and includes a self-lubricating element, the method comprising a step in which an element which is not self-lubricating is modified so that it becomes the self-lubricating element.

### DESCRIPTION OF FIGURES

FIG. 1 illustrates an endoscopic apparatus of the state of the art.

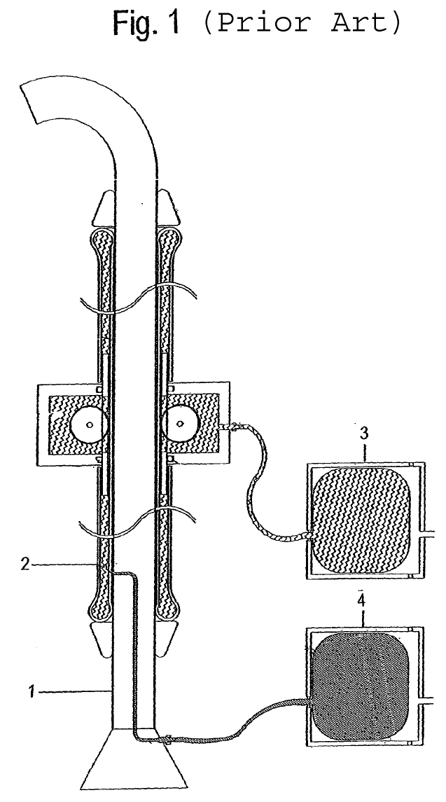

FIG. 2 illustrates another endoscopic apparatus of the state of the art.

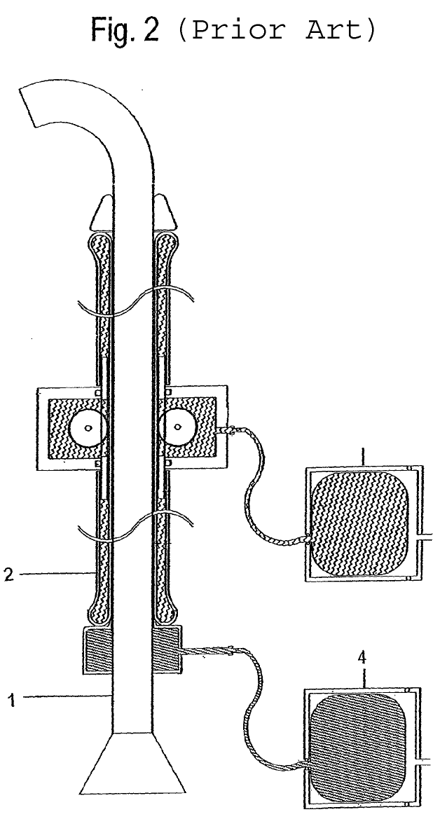

### PREFERRED EMBODIMENTS OF THE INVENTION

The invention provides a medico-technical device adapted to be engaged with a human or animal body comprising a self-lubricating element exposed to internal friction in the device.

A self-lubricating element in accordance with the present invention is an element which can develop a lubricating effect without supply of a lubricant from outside. By lubricating effect especially the reduction of the friction coefficient during friction between the self-lubricating element and a counter-element, the latter being in contact with the self-lubricating element, as well as during friction between different sections of the self-lubricating element is understood.

The self-lubricating characteristics permit a significant reduction of the internal friction, especially the friction between different sections of the self-lubricating element and between the element, on the one hand, and other parts of the medico-technical device, on the other hand.

Moreover, in contrast to an external supply of lubricant the structure of the device is simplified and the handling of the medico-technical device is considerably improved. The self-lubricating characteristics of the element in the device per se further enable the device to be designed as throw-away article which is easy to manufacture and cheaper. Furthermore, the consumption of lubricant is reduced, which has further economic advantages.

Also the phenomenon occurring in prior art that lubricant gets into the examined human or animal body can be most largely or totally prevented by the device according to the invention.

Especially non-vulcanized or vulcanized silicone rubber, PTFE, ePTFE and polyurethane(s) are considered as particularly good materials for the self-lubricating element, because they are good to work, may already have self-lubricating properties as such and exhibit very good mechanical properties such as impact strength, bending behavior, etc., which are appropriately compensated especially when using the self-lubricating element as a tube, in particular as everting tube. Said materials can be mixed with further materials.

In accordance with a preferred embodiment of the invention, the material which forms a substantial part of the self-lubricating element includes one or more rubbers selected from the group consisting of silicone rubbers, natural rubbers and (co)polymeric rubbers.

Suitable (co)polymeric rubbers are rubbers from copolymers which comprise at least one, usually two, three or more different monomers.

Moreover, rubber compositions of the present invention may contain suited softeners, fillers and/or foaming agents and the like.

Silicone rubbers can be especially advantageously used as basic substances of the rubber element according to the invention and are therefore preferred.

When using a silicone rubber, the rubber element possibly can contain hydrophobing agents, in addition to the afore-described additives. They prevent absorption of water and, as a consequence, that absorbed water results in a separation of chain elements and thus in undesired modifications of the silicone rubber.

For instance, silicone compounds containing silanol and/or polyalkyl disilazanes can be used as hydrophobing agents.

As further additives rhodium and/or platinum catalyst(s) and, where appropriate, one or more inhibitors can be additionally added. For an additive vulcanization especially platinum catalysts are suited, preferably Pt(0) complexes which can include e.g. vinyl siloxanes as ligands.

As possible inhibitors specifically acetylenic alcohols, such as e.g. 2-methylbutinol and ethinylcyclohexanol, tetramethyltetravinylcyclotetrasiloxane or tetramethyldivinyldisiloxane can be indicated.

If the silicone rubber is peroxidically vulcanized, preferably alkyl or aryl peroxides such as e.g. dicumylperoxide, 1,4-bis(tert-butylperoxo)-1,4-dimethylhexane, 2,4-dichlorobenzoylperoxide and 4-methylbenzoylperoxide can be added instead of the catalyst.

Furthermore, for medico-technical applications also halogen-containing, especially fluorine-containing polyolefins are suited, because they are often inert and consequently show very good body compatibility. By virtue of its inert and biocompatible characteristics and the ready accessibility, PTFE is most preferred, either as normal PTFE or in expanded form (ePTFE).

Advantageously the self-lubricating element can be obtained by surface treatment of an untreated element so as to form and/or intensify its self-lubricating properties. Suited methods of surface treatment are, for instance, silicating, ultraviolet radiation, ozone treatment, texturing by photo lacquer or screen printing techniques and a corona treatment.

It is also possible to tailor-make further layers, for example by centrifugal, dip or spray coating, possibly polymerization of an applied solution by means of e⁻ or γ rays, graft polymerization by photo-coupling, ceramic coating, acrylate coating or aryl coating. Suited methods for applying coatings for surface treatment inter alia are, in this context, sputtering, PVD (physical vapor deposition) and CVD (chemical vapor deposition) as well as powder coating.

As further method plasma treatment has to be mentioned which permits various possibilities of surface treatment, e.g. in response to the material to be treated, and can cause particularly good self-lubricating properties. Moreover, plasma treatment is very interesting in the economic respect, because large unit numbers can be manufactured at low cost in comparatively short time.

Plasma is understood to be an ionized gas having an equal number of positive and negative charges. Typically used gases are especially O₂ and F₂ as etching gases by which the surface of the workpiece can be cleaned; inert gases such as argon as well as hydrogen which can cause vulcanization through ultraviolet effect as well as fragmenting and repeated separation; NH₃, N₂, SO₂, air and water which permit functionalizing and thus activating the surface; organic and metalorganic gases which can form a plasma polymeric layer by separation; as well as halogen gases; and mixtures of the afore-mentioned gases.

By all afore-mentioned gases radical locations can be produced which permit further treatments such as a post-vulcanization, post-oxidation and grafting with functional monomers which are found in the final product as polymer layer.

Moreover, quite specific functionalizations of the treated surface can be attained by the selection of the gases used. For instance, an ═OH group can be introduced by means of O₂, an OH group can be introduced by means of H₂O, H₂/O₂ or O₂, a COOH group by means of H₂O, H₂/O₂ or CO₂/H₂, an F group by means of CF₄, SF₆, BF₃, XeF₆, SOF₂, NF₃ or SiF₄, a Cl group by means of CCl₄ or BCl₃, an NH₂ group by means of NH₃, N₂H₄ or N₂/H₂ and a SH group by means of H₂S, H₂/S₈ or CS₂.

These gases can, by applying a high frequency, be transformed into their respective plasmas which then usually contain electrons, radicals, ions, ultraviolet radiation and, in response to the gas used, a plurality of different excited particles.

During surface treatment in the invention low-pressure plasmas are used, inter alia, e.g. having a pressure of 1 to 1000 Pa, which facilitates the exploitation of chemically extremely reactive plasma for surface treatment. Appropriate capacities for igniting the plasma, e.g. by glow discharge, are within the range of preferably 10 to 5000 W. Typical times of treatment range from several seconds to some minutes.

The actual surface treatment can take place directly in the plasma zone, outside the plasma zone (so-called “remote method”) or initially for activation in the plasma zone with subsequent graft reaction. It is also possible to coat the element to be treated initially with a reaction solution, especially a polymer solution, for instance by dipping and then to carry out a fixing step within the plasma zone.

The surface of the element can be modified by the plasma treatment in different ways. A first modification consists in cleaning and sterilizing by etching removal. During this etching operation the wettability of the surface is varied and especially the roughness thereof is increased. Concretely spoken, by material removal the micro-roughness of the surface is increased, i.e. a rough texture with irregularities is produced the average depth of which is, measured from the wave peak to the wave trough, within the range of preferably 1 to 2500 nm, more preferably 1 to 1500 nm and most preferably 1 to 1000 nm. Consequently, this is a plasma pre-treatment or activation to which further steps are connected in order to finally arrive at the self-lubricating element of the invention. It has turned out that a self-lubricating element according to the invention, especially made of silicone rubber, shows an improved self-lubricating property, when it has initially passed through the plasma activation.

The micro-roughness produced substantially facilitates the application of further substances or layers, especially of further lubricants according to the present invention. In a preferred embodiment silicone oil, especially hydrophiled silicone oil is applied which is fixed on the pre-treated surface in a subsequent second plasma method. The thickness of the silicone oil layer applied is preferably within the range of 0.1 to 1 nm.

A second modification consists in producing radical locations. A secondary reaction can be initiated by the formation of said radical locations, e.g. a further vulcanization of an applied, especially polymeric substance. In this way, the polymer network can be formed more densely, which on the whole improves the lubricating properties of the resulting surface.

This effect is further increased, when the radical locations are the starting point for a subsequent grafting reaction. A polymeric substance which is preferably different from the basic material of the self-lubricating element (e.g. silicone rubber or PTFE) is grafted onto the already treated surface. In this way, for instance vinylpyrrolidine, vinylimidazole, ethoxide (block)copolymers of polyethylene oxide and polypropylene oxide, allylic polyethylene oxide and (2-hydroyethyl)methacrylate can be grafted.

The radical locations formed are moreover suited for forming hydrophilic locations by reaction with e.g. oxygen, such as atmospheric oxygen, so that the treated surface in total is made hydrophilic.

According to the invention, preferred use is made of the so-called low-pressure plasma treatment. Low-pressure plasma treatment means that in a vacuum chamber, in which a very low vacuum of usually less than 0.5 mbar prevails, the tubes are inserted in order to be subsequently coated with e.g. siloxane or polycarbons by ionization with approx. 1000 volt at a temperature of 20 to 40° C., for instance. By the gas inlet, radicals are formed on the surface of the tubes in the above-described manner and polymers are formed of monomer silane molecules (polymerization).

In the case of a tube-shaped workpiece an external and internal coating can be carried out in a very simple manner. This can be done in one operating cycle by applying, in addition to the afore-mentioned coating, one/two electrode(s) to the end(s) of the tubes so that said electrode(s) ensure(s) that also the inner wall of the tubes is coated.

In addition to the low-pressure plasma treatment there is an atmospheric pressure plasma treatment, as it is called, which can likewise be applied according to the invention.

Moreover, the plasmas used are distinguished depending on the temperature used, mainly into a low-temperature plasma and a high-temperature plasma. The high-temperature plasma works with fusion plasmas, wherein the gas temperature is typically within the range of 10⁷ K. The low-temperature plasma is further subdivided into non-thermal and thermal plasma. The non-thermal plasma is produced, for instance, by low-pressure glow discharge, corona discharge or barrier discharge, the gas temperature typically ranging from 300 to 10³ K. Thermal plasma is produced, for instance, by arc discharge, arc ray plasma or an RF arc torch, the gas temperature typically being no more than 2*10⁴ K.

The time required for coating can be appropriately adjusted in response to the material of the workpiece, to the layer to be built and to the thickness thereof and is typically within the range of minutes, wherein also times of treatment of up to half an hour are taken into account.

In accordance with the invention, the following ionization methods are preferably employed: In one method, oxygen is used as gas so as to vary a submitted silicone material such that a smooth surface is resulting. In another method, siloxane is converted via a liquid preliminary form into a gaseous state; the same mode of operation is preferred for coating with fluorocarbon. The two latter variants have the advantage of an even smoother surface, which entails a definite reduction of the frictional forces, wherein even better results are obtained when the two methods are carried out one after the other.

For this reason, silicone tubes obtained by plasma treatment and coated with siloxane and/or fluorocarbon are especially preferred embodiments of the present invention. Apart from that, it is also preferred for an improved gliding property to form a PTFE or ePTFE layer by plasma coating on the self-lubricating element, especially on a self-lubricating silicone element such as a silicone tube. By this coating with (e)PTFE further reductions of the friction coefficient of the self-lubricating element can be attained.

Furthermore, it is possible by the aforementioned plasma polymerization (preceding application of polymeric solution followed by fixation in the plasma) to build up further layers having desired properties which in turn are capable of contributing to self-lubrication. With this layer formation it can be chosen, in turn, whether the surface shall have hydrophilic or hydrophobic properties.

Basically it is stated that an application-dependent specific setting of the surface of the self-lubricating element of the invention can be obtained by the surface treatment. I.e. the surface energy can be quite selectively adjusted in response to whether any further layers and/or lubricants are to be applied.

In the chemical respect it is advantageous in this context that the surface can be selectively equipped with hydrophilic or hydrophobic properties, which results in an improved lubricating effect, when two different sections of the element get into sliding contact, and moreover facilitates the application of further coatings in response to the hydrophilic or hydrophobic property thereof. In addition, also a selective activation of the surface by introducing active groups is accessible in this way. In order to obtain a hydrophobic surface preferably siloxane plasmas are used, especially siloxane perfluorocarbon plasmas. In order to obtain oil-repellent properties grafting with acrylates, especially perfluoroacrylates is preferably taken into consideration.

Adjusting the surface to either a hydrophilic or a hydrophobic surface is dependent in particular on a lubricant which is to be optimally used as a supplement. For instance, when the surface of the self-lubricating element is hydrophilic, water or an aqueous solution and/or an aqueous mixture can be used as supplementary lubricant. Exactly in the case of endoscopic applications in which frequently oily sliding agents have conventionally been used, which are not tolerated or accepted very well by the individual examined, the use of water or aqueous solutions/mixtures results in definite improvements.

If it is necessary or desired, on the other hand, to make supplementary use of oily lubricants, there is a particularly good compatibility with the self-lubricating element and thus an improved gliding effect is resulting when the surface of the self-lubricating element is rendered hydrophobic.

An advantageous coating on the surface of the element as such already significantly reduces the friction properties and thus contributes to the self-lubricating characteristics of the coated element. In order to ensure surface properties suited for application it may be advantageous to first subject the element to a chemical, mechanical or physical pre-treatment, especially to a corona pre-treatment.

Appropriate coating materials which develop self-lubrication are especially graphite, nitrides such as boron nitride, fluorocarbons, MoS₂, WS₂, talcum, silicone oil, (poly)silazane, (poly)siloxane, PTFE and ePTFE. Moreover, a combination of a polysaccharide and a protein, especially a combination of alginate with whey protein, soy protein etc. as well as with mixtures of such proteins, the gliding properties and biocompatibility of which are excellent, has turned out to be a particularly advantageous coating. In addition, it has proved to be advantageous to build up a coating of gelatin, agar-agar and/or cellulose to which further lubricants, especially triglycerides, can be added as an option.

The coating materials are provided, for instance, as nanoparticles or macromolecules. However, the coating materials may also be provided as micro particles or macro particles.

The surface structure can be adjusted in a well-directed manner by the use of the coating materials in the form of particles such as nanoparticles, for instance by a layer-by-layer build-up. The particles can be provided on the treated surface particularly in the form of self-organized mono layers, as particles having a micro-texture or as nanoparticles covered with surface-active agents.

Some further methods of surface treatment preferred in the invention, i.e. for a mere coating or surface modification or for smoothing after completed engraving (described hereinafter) will be described in detail in the following.

In the case of ceramic coating a thin ceramic layer (ceramic film) is applied to the surface on which cavities are optionally formed at low temperature by a conventional coating method for ceramic coating. The treated surface can be smoothed by separating a ceramic film, in addition the biocompatibility is increased.

On principle, inorganic ceramic or glass coatings can be applied to the surface of the element by the methods known in engineering. Starting substances, which result in especially suited coatings for medico-technical applications, are silanes, halogenized silanes, especially fluorinated silanes, and silicates on the basis of water glass as well as network structures of SiO₂, Al₂O₃ and/or TiO₂ that are accessible by the sol-gel method.

Silicating of a surface (including an engraved surface) is performed such, for instance, that a silicon-organic compound (silane) is fed into a flame. Then a thin though very dense and strongly adhering silicate layer which is stable to moisture and frequently has a high surface energy is produced on the treated surface by a so-called flame pyrolysis. Due to the short exposure time of the flame the surface temperature of the workpiece can be kept very low. Therefore, this method is suited even for very thin-walled synthetic materials.

The silicate layer practically adheres to all surfaces and forms a nano-porous surface structure which ensures an optimum chemical bonding of organic components, especially e.g. of a lubricant, to the silicate layer.

As an alternative it is possible, when using a silicone rubber as material for the element exposed to internal friction, to thermally transform the uppermost silicone layer into a silicate layer by a plasma method. In this way the uppermost layer is glass-like hardened so that the advantages of an improved hardness and a definitely smoother surface result.

If a silicone rubber is used as material for the element exposed to internal friction, even further possibilities of an advantageous and friction-reducing coating of the profiled or non-profiled surface are resulting. The silicone rubber shows on its surface OH groups which can be chemically transformed such that hydrophilic or hydrophobic properties are imparted to the rubber.

It is also possible that said materials used for coating, especially graphite, are mixed in the entire element constituted of silicone rubber. It may be expedient in this context that suited agents are added which increase the compatibility between silicone, on the one hand, and the material mixed therein, on the other hand. An example of such an agent is, for instance, dimethyl disulphide (DMDS) which increases the dispersing capacity especially of graphite in the silicone rubber.

When the element exposed to internal friction itself is formed of PTFE or ePTFE, likewise coatings including the aforementioned materials can be advantageously employed. Moreover, also in this case it is possible that said materials, e.g. graphite, are mixed in the entire element formed of PTFE or ePTFE. In this event, it may also be favorable for improving the dispersion state that in addition agents and other additive substances are added, for instance appropriate emulsifiers.

The coatings produced as described above including (or the addition of) e.g. graphite, fluorocarbon, MoS₂, WS₂, talcum, silazanes, siloxane, PTFE, ePTFE and silicone oil and/or alginate+protein, especially alginate+whey protein and/or alginate+soy protein, further reduce the friction and thus reduce the abrasion during a longstanding use.

In case that a comprehensive abrasion of the coating, for instance of PTFE, occurs in the course of use, the coating forms a so-called sacrificial layer. The element according to the invention then is preferably designed such that even after partial or complete consumption of the sacrificial layer an emergency running property is retained, i.e. a reduced self-lubricating property which still permits the use, however.

A modification of the surface by ultraviolet radiation is likewise possible, wherein the ultraviolet radiation is preferably carried out in a vacuum. The surface to be treated is radiated with ultraviolet light, i.e. light having a wavelength of approx. ≦400 nm, under fixed pressure in a defined atmosphere, e.g. a nitrogen or nitrogen/oxygen atmosphere, during a fixed period of time. Typical radiation times adapted to achieve a sufficient smoothing range from 0.5 to 20 minutes, especially 1 to 2.5 minutes, wherein from a radiation of approx. 20 minutes or more usually there is no further improvement so that such long radiation times are economically disadvantageous. Therefore, according to the invention preferred radiation takes place for a period of 0.5 to 20 minutes, shorter radiation times of e.g. 1 to 2.5 minutes being of advantage to obtain an especially good wettability. Typical doses of radiation are within the range of from 2 to 40 J/cm². Ultraviolet radiation in a vacuum (VUV method) can be employed in a particularly advantageous manner, when the element exposed to friction includes silicone rubber at least on its surface and is preferably made of silicone rubber as a whole. The reason is that in this event a subsequent vulcanization of the silicone surface occurs which smoothes the latter and avoids or definitely reduces adhesive effects.

The advantages of ultraviolet radiation reside especially in the low expenditure on apparatuses as well as in the fact that neither problems of compatibility nor problems of adhesive strength occur.

In the case of acrylate coating, the surface to be treated is coated with an acrylate layer similar to a color application. For this purpose, dip coating, roller coating, doctor coating, spin coating, the so-called doctor-blade method etc. can be advantageously used to obtain a uniform and thus especially smooth coating. After coating a hardening can be performed, where appropriate. It is especially expedient in this context to carry out e.g. a hardening by ultraviolet radiation. The acrylate coatings produced expediently show a thickness of from 2 to 15 μm, smaller coating thicknesses of from 2 to 10 μm, especially 2 to 5 μm being preferred under the aspect of flexibility.

Each acrylate known in engineering can be used as acrylate for this purpose. The suited selection of the acrylate permits to adjust the properties of the surface of the element exposed to internal friction.

Moreover, it is possible to perform a smoothing of the profiled surface by producing a polysilazane layer, either an organic or an inorganic polysilazane layer. Such polysilazane layer can be applied of advantage by dip coating and subsequent oxidative transformation. Perhydropolysilazane, for instance, is applied and oxidized by dipping. In this way, also very thin coatings are adapted to be formed which are advantageous to the flexibility of the coated element, for instance having a thickness of from 0.01 to ≦1 μm.

It has further turned out that in this way very good adhesive strengths can be achieved, wherein less problematic, i.e. no or hardly irritating or etching substances are used, which can strongly improve the approvability in the individual case in medico-technical applications. In addition, the product range of polysilazanes is very wide so that raw materials exactly adapted to the use and the desired properties can be selected.

The afore-mentioned aryl coating is advantageously carried out by the CVD method, aryl compounds being used as precursor. Examples of precursor compounds are substituted benzenes such as o-, m- or p-xylene or else di-p-xylenedimer.

As organic coatings especially well suited for medico-technical applications are moreover mentioned 2-component expoxide burn-in coatings, polyurethane burn-in coatings, 2-component-polyurethane air-drying lacquers as well as fluorinated polymers. For the fluorinated polymers polytetrafluoroethylene, fluorinated ethylene/propylene-copolymer, perfluoroalkoxy copolymer, polyvinylidene fluoride and ethylenechlorotrifluoroethylene are especially well suited.

In order to selectively adjust a hydrophilic surface a polyacrylonitrile coating is furthermore advantageous, while a hydrophobic surface can be selectively adjusted by means of a polysulphone coating.

Concerning the above-listed coating techniques, it is further of advantage that they are preceded by a pre-treatment. Especially good adhesive strength and gliding behavior are achieved when in particular corona activation, fluorination or flame treatment are used as pre-treatment, wherein corona activation provides the best results.

The surface energy of the resulting surface, which is finally exposed to the friction, can also be adjusted by a specific coating of the basic element with a further substance, i.e. by the build-up of one or more layers on a possibly pre-treated surface.

The self-lubricating element may also consist of a not self-lubricating basic element and a lubricant added to the latter. Also, for a further reduction of the friction resistance another lubricant may be added to one of the afore-described self-lubricating elements.

In order to ensure particularly good separation of lubricant as well as storage of lubricant directly on the surface of the self-lubricating element, it is preferred that at least one surface of the self-lubricating element includes a profile the cavities of which contain a lubricant. It is especially preferred that such cavities are additionally formed in a self-lubricating element which has been subjected to the above-described plasma treatment after forming the cavities, preferably a self-lubricating element consisting of silicone, (e)PTFE or polyurethane and provided with a siloxane or perfluorocarbon coating that is formed by plasma treatment, the cavities being covered with the coating.

Such a profiled (i.e. provided with cavities containing lubricant) self-lubricating element can be employed especially advantageously as everting tube and/or shaft sheathing for an endoscope, in particular for examination of the small intestine. The cavities form so-to-speak a reservoir for additional lubricant which then obtains better, i.e. reduced friction by residual lubrication in the close turns.

This can also be achieved, inter alia, by fat applied during manufacture which is provided in the cavities as well as optionally in addition outside the latter and which becomes more liquid at body temperature than outside the body. In contrast to a lubrication of the shaft and/or everting tube system of the endoscope carried out before and/or during the examination, a single filling of the cavities is by far easier than lubrication necessary during the examination.

Said cavities forming the profile can have a regular or irregular shape. The depth of the cavities formed is approximately 10 to 250 μm, preferably 50 to 200 μm and especially preferred 100 to 150 μm. In the case of a too small depth the amount of lubricant absorbed can be insufficient, if the cavities are too deep, disadvantageous damage of the edges of the cavities is easily caused during formation of the latter. In order to provide a good compensation the preferred depth is within the given range. For the same reason, the cavities preferably have a diameter of from 50 to 250 μm, more preferably from 100 to 200 μm, further preferred from 130 to 170 μm and especially of about 150 μm.

On principle, use can be made of all lubricants described here, wherein lubricants on vegetable basis are preferred, in particular palm fat, as it is well tolerated, is furthermore easy to handle and can be easily filled into the profile.

When cavities containing lubricant are provided on at least one surface of the element, it is possible that the cavities release lubricant due to external influences in a quite selective manner and at a predetermined point in time. The cavities, e.g. in the form of grooves, can be advantageously produced by embossing, foaming and cutting open, cutting or rolling or by engraving, especially laser engraving.

Therefore, in a preferred embodiment of the invention the lubricant is therefore provided in grooves that are directly arranged on a surface of the element. In some configurations regularly arranged grooves, in other configurations irregularly arranged grooves constitute the profile. Preferred embodiments of the profile will be described hereinafter in detail.

The afore-mentioned profile is preferably adapted so that released lubricant is supplied to the areas exposed to especially high friction. For this purpose, the profile may have longitudinally, transversely or spirally extending cavities that are preferably formed by grooves or by individual locally confined indentations.

Often, in different applications also two separated or different sections of the self-lubricating element get into contact with each other, which results in an increased friction at this location. Therefore it is especially advantageous when the profile is adapted such that upon superposition of two opposing sections of the self-lubricating element the respective profile sections cannot engage.

Consequently, in the event of a tube-shaped element preferably grooves are provided which enclose an angle of more than 0° to less than 90° with the surface line of the tube-shaped element.

It has turned out to be especially suited for the present invention that the profile shows a preferred depth of from 50 to 500 μm and more preferred from 200 to 400 μm. With a depth of less than 50 μm, only a restricted amount of lubricant can be absorbed and the self-lubricating effect subsides relatively quickly. On the other hand, depths of more than 500 μm easily result in poorer dimensional accuracies during manufacture. Moreover, when forming such profile of deeper than 500 μm, there are stronger (e.g. mechanical or thermal) impacts on the treated surface so that larger irregularities are susceptible to be formed on the surface which reduces the gliding effect. Therefore, the range of from 50 to 500 μm is preferred in the present invention, the described drawbacks being even further reduced within the range of from 200 to 400 μm.

A profile especially suited for the purpose of absorbing lubricant and discharging lubricant in a controlled manner can be produced by engraving, in particular by laser engraving. Therefore it is preferred that the profile is adapted to be produced by engraving by laser. A self-lubricating element having a profile adapted to be produced by laser engraving is thus likewise included in the present invention.

Laser engraving permits a controlled material removal, for instance by means of a pulsated laser beam. The beam is preferably focused very strongly to improve the machining accuracy, wherein e.g. focal points having a diameter of 0.01 to 0.1 mm are generated. Extremely high temperatures of more than 2000° C., for example, can be reached in the focal point, which permits rapid working of almost any material.

Since the material removal takes place on the basis of thermal processes, the hardness of the material to be worked is of minor importance. Therefore, the method can be applied to all materials usable according to the invention. Furthermore, compared to other applicable methods, such as e.g. eroding, the time saving is up to 60%.

During laser engraving the material to be removed is melted and evaporated only locally, which permits very precise working. The precision depends, inter alia, on the focal diameter of the focused beam and on the accuracy of the laser. Therefore, both parameters are preferably set to values of ≦0.1 mm, according to the invention.

The wavelength of the laser likewise plays an important role for the material removal, i.e. to which extent the radiated energy is absorbed. The degree of absorption in turn is dependent on the material and on the wavelength of the laser used. Consequently, the selection of appropriate wavelengths is very important and is preferably selected in the present invention such that the machined material absorbs at least 50% or more, more preferably at least 60% or more and especially preferred at least 70% or more of the laser energy.

In accordance with the invention, solid lasers, for instance, are employed for laser engraving. Said lasers can operate lamp-pumped or diode-pumped. Usually their original wavelengths are within the IR range, but they can also be brought up to the ultraviolet range (UV) by means of specific optical crystals. This is advantageous both because of the absorbing characteristics of different materials and because of the focusing capacity. Compared to IR laser beams, UV laser beams can also be focused on smaller focal diameters.

Apart from that, the use of an excimer laser or a CO₂ laser is particularly preferred for an especially filigree working which is easy to control. Laser working by a CO₂ laser is cheaper and thus more advantageous. The CO₂ laser preferably operates at a wavelength of 10600 nm. The pulse energy is set, of advantage, between approx. 10 and 100 μs and especially between 20 and 60 μs.

During an engraving step wave-like corrugations may occur on the treated surface on the side of the produced cavities. Such corrugations reduce the self-lubricating properties of the element. Therefore, it is furthermore preferred to obtain the profile by laser engraving and subsequent smoothing.

The methods already described in the foregoing have proved to be especially suited techniques for smoothing the engraved surface so that the wave-like corrugations are levelled and simultaneously a depth of the engraved cavities suited for absorbing a sufficient amount of lubricant is maintained. So, especially ceramic coating, silicating, ultraviolet radiation, acrylate coating, aryl coating, polysilazane coating and polysiloxane coating can be employed for quickly and uniformly achieving the required smoothing. With suited times and conditions of treatment also a plasma treatment may be adapted to bring about a smoothing, especially when the plasma polymerization or a grafting of polymer(s) after a plasma treatment is concerned.

In another preferred embodiment of the invention, the self-lubricating element has a layered structure. The layered structure comprises at least one substrate layer so as to form a stable basis for layers disposed thereon, a reservoir layer for lubricant disposed on at least one, preferably both sides of the substrate so as to store sufficient lubricant for the actual use, and at least one or, if on both sides of the substrate a reservoir layer is disposed, two external perforated layer(s) so as to discharge the lubricant in a controlled manner. In the first case, the basic structure consists of substrate/reservoir layer/perforated layer, in the second case the basic structure consists of perforated layer/reservoir layer/substrate/reservoir layer/perforated layer, wherein respective intermediate layers can further be provided.

The substrate layer can be made, e.g., of one of the already afore-mentioned rubbers, silicone rubber being especially preferred due to the stated advantages. The substrate layer is designed such that it is substantially impermeable for the lubricant used.

The reservoir layer can be formed so that the lubricant is in solid form as lubricant layer. The lubricant then is adapted such that it softens or liquefies due to influences from outside, as described hereinafter, and is discharged to at least one neighboring layer.

Moreover, it is possible that the reservoir layer has a sponge-like structure or is formed by a material having a capillary effect, wherein the lubricant is sucked in by the sponge-like structure and/or by the material and is subsequently, or when specific outer influences occur, discharged to at least one neighboring layer.

In the event that two reservoir layers are provided they can have the same or a different shape. A different design can be especially advantageous when different pressure influences are given. If the self-lubricating element according to the invention is in the form of an everting tube, for instance, especially in the eversion area different physical influences occur, such as pressure in the inner and outer reservoir layers, so that a different design of this layer adapted to the respective external conditions may be useful. The same consideration is also applicable to the perforated layer described hereinafter. The perforated layer is preferably provided directly adjacent to the reservoir layer and forms the outer surface of the element. However, between the reservoir layer and the perforated layer further layers can be disposed as long as they are substantially permeable for the lubricant. The perforation of the perforated layer may have any design. It is preferred that this layer has through holes or ducts the central axes of which enclose an almost right angle with the surface of the element formed by the perforated layer, i.e. of about 80 to 100°, especially of about 90°. In this way, the lubricant gets from the reservoir layer to the surface to be lubricated in an especially effective and controlled manner.

Moreover it is possible to form the perforated or porous layer by one of the above-described plasma methods.

The layer structure of the element can be easily manufactured by producing, e.g., the individual layers in advance as required and then laminating them together or by applying the lubricant layer onto the substrate or onto the uppermost of layers possibly arranged above the substrate and then providing this compound with a perforated layer. It is an advantage of this embodiment that the lubricant reservoir can be designed to have any use-dependent size.

During the internal friction of the medico-technical device lubricant is released e.g. by physical or chemical influences. The physical influences include, for instance, pressure and temperature influences or variations. Pressure influences occur especially when the medico-technical device adapts to body conditions during its use. Such a situation typically appears in endoscopy when the distal end, i.e. the end engaged with and/or inserted in the body, is bent during propulsion.

As examples of chemical influences acid (pH=0 to 6.99) and basic (pH=7.01 to 14) solutions can be mentioned which are adapted to be applied to the counter-element e.g. by immersing into the solutions. The acid or basic solutions result in a chemical modification of the lubricant having an influence on the softening of melting point thereof, i.e. reducing the same, for instance, so that lubricant escapes.

So that possibly no or no excessive reaction occurs between an acid and/or basic solution and the material, the pH values thereof should preferably range from 5 to 6.99 (acid solution) and/or 7.01 to 9 (basic solution). Under physiological aspects, in medico-technical applications a pH of from 5 to 6.99 is preferred most.

The chemical influence moreover need not necessarily be exerted by acid of basic solutions. It is sufficient for this embodiment that lubricant is released by the chemical interaction.

It is further preferred that the element exposed to internal friction is made of a material comprising a rubber and a lubricant. The use of a rubber and/or a rubber mixture permits an especially flexible configuration of the element, which is advantageous especially with regard to the medico-technical use, preferably in the field of endoscopy.

By reason of its easy workability and its economic efficiency a silicone rubber is preferably used for the rubber. Moreover, a high dimensional accuracy can be achieved especially easily by laser engraving, if use is made hereof. This has an advantageous effect on the gliding properties. The lubricant is suitably silicone oil, because it is chemically inert and its properties can easily be adjusted with respect to the application. Due to the high compatibility between silicone rubber, on the one hand, and silicone oil, on the other hand, this combination is an especially preferred combination of rubber and lubricant. When a profile is produced by engraving, use is preferably made of vegetable lubricant, e.g. palm fat.

As mentioned in the foregoing, the self-lubricating element of the medico-technical device preferably comprises self-lubricating rubber and a lubricant. The rubbers may have any design. As a rule, they are comparatively inexpensive and therefore permit mass production, especially of throw-away articles. Further the properties of the rubbers, such as e.g. the absorbing capacity for lubricants, flexibility, dimensional stability at increased temperatures etc. can be adjusted without difficulty.

Especially oily and gel-like lubricants such as oil-in-water emulsions (O/W emulsions), water-in-oil emulsions (W/O emulsions) and nitrogen emulsions are taken into consideration as lubricants.

Basically all types of oils are suited, such as vegetable oils, animal oils, mineral oils and synthetic oils.

Paraffin oil, especially viscous paraffin (in Latin paraffinum subliquidum) and glycerin are especially well suited lubricants, because usually they are well tolerated by human beings and many animals. In a preferred embodiment palm fat is used by virtue of its availability, its good lubricating properties and good workability as well as the excellent biocompatibility. Palm fat is advantageous especially in case that a profile is produced and filled with lubricant.

Also silicone oils are excellently suited, because they can be very well stored in particular in rubbers on silicone basis, as described in the foregoing, and can be discharged by said rubbers in a controlled manner so that a continuous lubricating effect can be ensured. Particularly advantageous gliding properties are obtained when silicone oil mixed with graphite is employed. In the case of an above-described coating of the silicone rubber element with PTFE a silicone/water emulsion is preferably used, as this can improve the interactions between the lubricant and the coating.

The medico-technical device may comprise a lubricant in various ways. In a preferred embodiment of the present invention the lubricant is spread almost homogenously in the material constituting the self-lubricating element. This is possible, for instance, by the fact that the lubricant is added when producing the material, especially rubber, and is mixed with the same or the starting substances thereof. Silicone rubber which further contains silicone oil is a typical example of such material.

In the afore-described case in which the lubricant is spread almost homogenously in the material, there is usually a certain incompatibility between the lubricant, on the one hand, and the material, on the other hand. This incompatibility is based on a difference in the chemical potential between the lubricant and the material. In this way a progressive separation takes place, comparable to a separation between an oil phase and a water phase. The separation causes the lubricant to “bleed” or to “exude” so that a lubricant film is formed on a surface of the self-lubricating element of the medico-technical element.

The advantages of this embodiment reside in the simple manufacture, the wide range of possible starting materials and ingredients as well as the continuous lubrication over a long period of time.

The above-described separation of lubricant and material is resulting in an increasing inhomogeneity of the lubricant in the material.

If short-term strong bleeding (exuding) of the lubricant is desired, it can moreover be advantageous that the element exposed to the internal friction is porous. Lubricant contained in the element then can get more quickly onto the surface of the element, for instance when the external pressure is increased, and can significantly increase the lubricating performance there.

It is of advantage in a similar way for a comparatively short but intense bleeding of lubricant, when the viscosity of the lubricant decreases at the actual temperature of use. Although such decrease is not specifically restricted, reductions of viscosity by at least 2.5%, preferably 5%, more preferred 7.5% up to 10%, 20% or even 50% or more are especially advantageous. In this case, for instance the so-called Mooney viscosity of the lubricant can be referred to, e.g. ML₁₊₄ (100° C.).

It is preferred for the lubricant that its temperature-viscosity characteristic exhibits a discontinuity in the temperature range of 10-43° C. In the medico-technical use, for instance in endoscopy, the element can be stored in a commercially available refrigerator, for example, and upon actual use, for instance at room temperature or at temperatures prevailing in human or animal bodies, develops an increased lubricating effect due to the increased fluidity of the lubricant.

Therefore, it is preferred when a definite reduction of viscosity occurs at room temperature, for instance between 10 and 30° C., preferably between 20 and 25° C. In other words, it is preferred that the temperature-viscosity characteristic of the lubricant exhibits a discontinuity within the temperature range of 10-43° C., preferred between 20 and 25° C., for instance a jump, especially a jump responsible for one of the afore-mentioned percentage variations.

It is equally very advantageous when the above-described decrease of viscosity of the lubricant occurs at temperatures usually prevailing in human or animal bodies. Consequently, for some embodiments a decrease of viscosity to the afore-described extent is desired within the temperature range of from 30 to 43° C., preferred within the range of from 36 to 38° C., especially around 37±0.5° C. That is to say, the lubricant exhibits a discontinuity in its temperature-viscosity characteristic in said temperature range, wherein again the afore-mentioned percentage variations are especially preferred.

In the present invention, preferred melting points for the lubricant used can also be within the range of 50° C., at approx. 50° C. or more. It is also preferred that the lubricant does not yet completely melt at body temperature, approx. 37° C., but the liquefaction only starts at this temperature.

Of course, also in said cases of a loss of viscosity at predetermined temperatures the element can be porous, which further increases the said advantageous effects.

The materials used for the self-lubricating element, especially rubbers, frequently can be equipped with an even better dimensional stability by a reinforcing agent contained therein. It is possible in this case that the reinforcing agent is impregnated with a lubricant, which likewise permits efficient storage of the lubricant in the material.

A reinforcing agent can, of course, also be provided in the above-described embodiments of storing the lubricant, wherein in said embodiments it can equally, but need not, be impregnated with lubricant.

Preferred examples of reinforcing agents are textile compounds that may be woven or non-woven. The textile compounds can have textile fibers which are either aligned almost in parallel in one or more directions or which are randomly disposed. The textile compound may also be provided substantially in the form of particles or powder.

Wool and cotton in various configurations are suited particularly well as materials for the textile compounds, especially when there is a high compatibility between the lubricant, on the one hand, and the cotton/woolen textile compound, on the other hand. In response to the chosen lubricant it may moreover be preferred to make use of synthetic textile compounds, preferably nylon-type, Dralon-type and/or rayon-type synthetic textile compounds.

Another example describes that on one or more surfaces of the element a lubricant layer is formed which softens by the action of pressure and/or heat and develops a lubricating effect. In this way, the self-lubricating element may consist of any suitable material adapted to be coated with a lubricant layer in a simple manner. Said lubricant layer is renewable so that also repeated use is possible.

It is furthermore possible and preferred that the self-lubricating element is in the form of a sponge or is constituted by a material developing a capillary effect. In this case, the lubricant is sucked in by the element and is held in cavities, for instance pores or ducts, by chemisorption or physisorption, for example. Furthermore, a material forming the self-lubricating element can be manufactured jointly with lubricant contained therein, especially in the event of rubbers, and cavities can be formed subsequently by foaming, for instance. Such sponge-like structure and/or such material exhibiting a capillary effect is suited especially well also for the above-described reservoir layer of the likewise above-described laminate.

By exerting pressure, for instance, upon such element filled with lubricant, the lubricant can be released again so that in turn a lubricant film forms on one or more surfaces of the element and thus self-lubrication takes place.

The self-lubricating element of the medico-technical device of the invention basically can have various shapes and, for instance, can be a rectangular or circular disk, a rectangular or multi-edged tube, a spiral or a hose. In a lot of applications, e.g. in endoscopy, it is preferred that the element is pipe-shaped or tube-shaped, however.

In the case of a tube-shaped self-lubricating element it is possible that only the outside of the tube has self-lubricating properties, that only the inside of the tube has self-lubricating properties or that both the outside and the inside of the tube have self-lubricating properties. It is preferred and of advantage that both sides have self-lubricating properties in particular when the tube is an everting tube, as it is called, which is everted at one side and thus comes to rest on itself. In so doing, a portion of the outside is sliding along another portion of the outside. If another element, e.g. an endoscope shaft, is introduced into the tube, the latter is propelled upon the relative movement of the tube. Apart from the reduced friction between the portions of the everting tube itself, moreover the friction with the inserted element, for instance the endoscope shaft, is reduced, wherein said shaft may itself be further covered, for example with another tube. Moreover, also the friction between the everting tube, on the one hand, and its environment, namely usually the walls of the body opening, is reduced.

If a lubricant additionally supplied from outside (e.g. oil) is used, it can be provided, on the one hand, between the self-lubricating element and the shaft or the exterior and, on the other hand, in the space formed between two tube portions upon eversion.

Typical lengths of such everting tube (in the unwound state) are within the range of 3 m, usually 3.20 to 3.50 m. However, in particular applications, especially in endoscopy of the small intestine, also lengths exceeding 3.50 m can be required. The lengths indicated here especially refer to examinations of human beings. Therefore, in veterinary applications appropriately adapted, i.e. definitely shorter but also longer lengths have to be taken into consideration.

It is further advantageous to endoscopic and other applications, if the self-lubricating element according to the invention is bendable or flexible, respectively. The element may have elastic or inelastic properties, the latter being preferred, because they permit better mechanical control of the device by virtue of the resulting dimensional stability.

The self-lubricating properties turn out to be advantageous during internal friction operations. Said friction operations can include friction between different portions of the self-lubricating element as well as friction between the element and a further counter-element.

A friction between two different portions of the element occurs in the case of an everting tube, as it is called, where a tube is wound such that an external surface seen from the center of the tube everts and gets into contact with itself.

Due to the respective advantages described in the foregoing, in an especially expedient manner the medico-technical device can be part of an endoscopic device, the self-lubricating element being in the form of an everting tube.

Preferably the self-lubricating element can be engaged with a human or animal body and gets into contact with the same. Since no excessive amounts of lubricant are discharged, the element behaves substantially neutral vis-à-vis the environment, in particular vis-à-vis the body tissue, so that this preferred configuration of the invention admits a contact between the device and the body tissue.

In a concrete, especially preferred configuration the self-lubricating element therefore constitutes an everting tube, in particular an everting tube for an endoscope (e.g. a coloscope, a jejunoscope, a gastroscope or a bronchoscope).

The device according to the invention may comprise a counter-element in addition to the element, wherein, in addition to the friction between different portions of the self-lubricating element, a friction between at least one surface of the element and at least one surface of the counter-element is caused.

The friction coefficient between the at least two opposing surfaces is considerably reduced by the self-lubricating properties of the element, which strongly improves the gliding capacity of the element and the counter-element against each other.

A further improvement of this gliding capacity can be achieved by designing the counter-element or a cover or sheath thereof in such a way as described in the foregoing for the self-lubricating element exposed to internal friction. For the counter-element each of the above embodiments can be used, wherein the element and the counter-element can be designed in the medico-technical device dependent on the circumstances of the intended use in the same way or in a different way. However, the counter-element and/or its cover can also be made of a material having no self-lubricating properties.

An advantageous embodiment consists in the fact that the surface of the counter-element is likewise provided with a lubricant. This lubricant can be adhering oil, a laminated lubricating film and/or a solid lubricant.

For the adhering oil the same oils can be used as indicated as examples of the lubricant in the foregoing.

The laminated lubricating film may consist, for instance, of the rubbers described in the foregoing as appropriate substances for the material.

The solid lubricant which may be advantageously provided on the surface of the counter-element can be either directly applied to the surface or can adhere to the surface by means of a binding resin. Such binding resin can comprise one or more of the afore-described rubbers.

Concrete examples of the solid lubricant preferred by virtue of their good gliding properties include carbon compounds such as graphite, nitride such as boron nitride, compounds containing fluorine such as fluorine resins and sulphides such as molybdenum sulphide and tungsten sulphide.

In a preferred embodiment, the counter-element is an endoscope shaft which is preferably inserted in an everting tube formed by the self-lubricating element and the surface or cover of which is preferably designed as a self-lubricating element according to the invention. The surface or cover, respectively, which itself is provided as tube, may naturally also consist of a material having no self-lubricating properties, as long as the self-lubricating element of the present invention is used as everting tube.

It is moreover advantageous and preferred that the lubricant employed according to the invention includes an active substance. A lubricant may contain an active substance without lubricating properties or the active substance itself has a lubricating effect and partly or completely forms the lubricant. In this way, in the medico-technical application respective useful active substances, in endoscopy especially analgesics, blood-stanching agents etc., can be locally and selectively employed and supplied.

In the present invention especially soothing agents, anti-inflammatory agents, decongestants, blood-stanching agents, disinfectants, humidifiers and analgesics are preferred, because when using the device according to the invention in an animal or human body, they cause the respective specific advantageous effects.

### EXAMPLES

### Example 1

A commercial silicone rubber was extruded to form an everting tube for an endoscope having a length of 3.20 cm.

Then grooves inclined with respect to the surface line of the tubes were engraved by a CO₂ laser having a wavelength of 10600 nm (pulse duration 40 μs). Grooves having a depth of 300 μm were formed.

The grooves were filled at 60° C. with palm fat liquid at this temperature (starting temperature of softening of the fat: >35° C.). After that, the tube was cooled and the palm fat solidified in the grooves.

The resulting tube was used as an everting tube for an endoscope. It turned out that the friction between the tube and the endoscope shaft as well as between the tube portions in the everting area was definitely reduced, an improved lubricating effect was brought about and at the same time hardly any lubricant escaped from the endoscopic device.

### Example 2

Two tubes were extruded as in example 1, one of which was engraved by the laser as in example 1. After that, both tubes were coated with a ceramic film. The grooves of the engraved tube were filled with palm fat, as in example 1. In this way, in both cases excellent gliding properties were achieved.

### Example 3

Two tubes were extruded as in example 1, one of which was engraved by the laser as in example 1. After that, both tubes were silicated on the surface according to a low-pressure plasma method. The grooves of the engraved tube were filled with palm fact as in example 1. In this manner, in both cases excellent gliding properties were achieved.

### Example 4

Two tubes were extruded as in example 1, one of which was engraved by the laser as in example 1. After that, both tubes were radiated with ultraviolet light for 10 minutes in a nitrogen atmosphere. The grooves of the engraved tube were filled with palm fat as in example 1. In this manner, in both cases excellent gliding properties were achieved.

### Example 5

Two tubes were extruded as in example 1, one of which was engraved by the laser as in example 1. Then both tubes were pre-treated by corona activation. After that, the tubes were coated with an acrylate layer by dip-coating. The grooves of the engraved tube were filled with palm fat as in example 1. In this manner, in both cases excellent gliding properties were achieved.

### Example 6

Example 5 was repeated with the exception that a fluorination was performed as pre-treatment instead of the corona pre-treatment. The results were substantially equal to those achieved in example 5.

### Example 7

Example 5 was repeated with the exception that a manual flaming was performed as pre-treatment instead of the corona pre-treatment. The results were substantially equal to those achieved in example 5.

### Example 8

Two tubes were extruded as in example 1, one of which was engraved by the laser as in example 1. After that, both tubes were coated with a perhydropolysilazane mixture by dip-coating. The coating was transformed into a polysilazane layer by oxidation at an increased temperature. The grooves of the engraved tube were filled with palm fat as in example 1. In this manner, in both cases excellent gliding properties were achieved.

### Example 9

The tests of the examples 1 to 8 in which the laser was used were repeated with the exception that a tube having a length of 3.50 m was employed, and wherein this time no grooves but substantially circular cavities having a depth of 100 to 150 μm and a diameter of 150 μm were produced. These cavities were filled with palm fat.

The everting tubes produced in this way were used in endoscopic examinations of the large and small intestines. It turned out that even with a deeper insertion into the subject to be examined a constant good lubrication could be maintained.

### Example 10

The silicone rubber employed in example 1 was extruded into eight everting tubes each having a length of 3.20 cm.

After that, four tubes were successively inserted in a reaction chamber (vacuum <0.5 mbar) for plasma treatment. Using O₂ as plasma gas, plasma pre-treatment was performed for several seconds such that the surface of the silicone tube was roughened on a micro level.

In a second step six tubes (three with pre-treatment, three without pre-treatment) were subjected to a low-pressure plasma treatment (vacuum <0.5 mbar; temperature=20-40° C.) to form a coating. In this way, two tubes at a time were coated with siloxane, with fluorocarbon and/or with ePTFE. All eight tubes were used as everting tubes for an endoscope shaft and the gliding properties were evaluated in accordance with the following criteria.

A: excellent gliding properties;
B: very good gliding properties;
C: still above-average gliding properties;
D: excessive friction.

The following table 1 shows the results of the evaluation:

TABLE 1

| | Plasma | | Gliding |
|---|---|---|---|
| Tube No. | pre-treatment | Coating | property |
| 1 | yes | — | C |
| 2 | no | siloxane | B |
| 3 | yes | siloxane | A |
| 4 | no | fluorocarbon | B |
| 5 | yes | fluorocarbon | A |
| 6 | no | ePTFE | B |
| 7 | yes | ePTFE | A |
| 8 | no | — | D |

### Example 11

Again tubes 1 to 7 according to example 10 were manufactured, provided with cavities by laser engraving as in example 9 and subsequently plasma pre-treated as in example 10, where appropriate (tubes 1, 3, 5 and 7) and then plasma-coated (not tube 1). The cavities were filled with palm fat. The evaluation was carried out analogously to example 10 and provided comparatively good results.

1. A medico-technical device adapted to be engaged with a human or animal body, comprising:
a self-lubricating element exposed to internal friction in the device, wherein at least one surface of the self-lubricating element is plasma-treated.

2. A device according to claim 1, wherein it is the self-lubricating element itself which is adapted to be engaged with a human or animal body and gets into contact with the body.

3. A device according to claim 1, wherein the plasma-treated surface is plasma-coated.

4. The device of claim 3, wherein:
the plasma-coated surface is coated with at least one of: siloxane, fluorocarbon, PTFE, ePTFE and polyurethane.

5. The device of claim 1, wherein:
at least one surface of the self-lubricating element is surface-treated by at least one of: ultraviolet radiation and silicating.

6. The device of claim 1, wherein:
the self-lubricating element exhibits at least one of: a ceramic coating, an acrylic coating and an aryl coating.

7. The device of claim 1, wherein:
the self-lubricating element includes silicone rubber.

8. The device of claim 1, wherein:
the self-lubricating element has a surface layer which includes a material selected from the group consisting of graphite, nitride, fluorocarbon, MoS₂, WS₂, talcum, silicone oil, polysilazane. polysiloxane, PTFE, ePTFE, gelatin, agar-agar, cellulose, and a combination of a polysaccharide and a protein.

9. The device of claim 8, wherein:
the surface of the self-lubricating element is hydrophilic.

10. The device of claim 8, wherein:
the surface of the self-lubricating element is hydrophobic.

11. A device according to claim 1, wherein at least one surface of the self-lubricating element includes cavities containing lubricant.

12. A device according to claim 11, wherein the cavities can be produced by laser-engraving.

13. The device of claim 11, wherein:
a plasma-coating is formed above the cavities.

14. The device of claim 11, wherein:
the cavities are adapted such that released lubricant is supplied to areas of the self-lubricating element which are exposed to higher friction than other areas.

15. The device of claim 11, wherein:
the cavities are adapted such that, when two opposed sections of the self-lubricating element are superimposed, the respective cavity sections cannot engage.

16. A device according to claim 1, wherein the self-lubricating element has a layered structure comprising at least one substrate layer, a reservoir layer for lubricants disposed on one or both sides of the substrate and one or two external perforated layers.

17. A device according to claim 16, wherein the substrate layer for the lubricant is substantially impermeable.

18. The device of claim 16, wherein:
the reservoir layer is formed so that the lubricant is provided in solid form as lubricant layer.

19. The device of claim 16, wherein:
the reservoir layer comprises a material having a capillary effect.

20. A device according to claim 1, wherein the self-lubricating element consists of a material in which a lubricant is spread in an almost homogenous manner.

21. A device according to claim 20, wherein there is an incompatibility between lubricant and material so that a progressive separation of the lubricant and the material takes place.

22. The device of claim 20, wherein:
the self lubricating element is porous.

23. The device of claim 20, wherein: the lubricant has a temperature-viscosity characteristic with a discontinuity within the temperature range of from 10 to 43° C.

24. The device of claim 20, wherein:
the self-lubricating element is tube-shaped.

25. An endoscopic device comprising:
the device according to claim 1, wherein the self-lubricating element is in the form of an everting tube.

26. (canceled)

27. A method of manufacturing a medico-technical device adapted to be engaged with a human or animal body and including a self-lubricating element, comprising a step of plasma treatment in which an element which is not self-lubricating is modified such that it becomes the self-lubricating element.

28-37. (canceled)

38. A medico-technical device adapted to be engaged with a human or animal body comprising an everting tube exposed to internal friction in the device, wherein it is the everting tube itself which is adapted to be engaged with a human or animal body and gets into contact with the body, and wherein at least one surface of the everting tube is plasma-treated and is coated with siloxane, fluorocarbon, PTFE, ePTFE and/or polyurethane.

39. A medico-technical device adapted to be engaged with a human or animal body comprising an evening tube exposed to internal friction in the device, wherein it is the everting tube itself which is adapted to be engaged with a human or animal body and gets into contact with the body, and wherein at least one surface of the everting tube is plasma-treated and is coated with siloxane, fluorocarbon, PTFE, ePTFE and/or polyurethane and wherein the everting tube is made of silicone.

40. An endoscopic device comprising an everting tube exposed to internal friction in the device, wherein it is the everting tube itself which is adapted to be engaged with a human or animal body and gets into contact with the body, and wherein at least one surface of the everting tube is plasma-treated and is coated with siloxane, fluorocarbon, PTFE, ePTFE and/or polyurethane and wherein the everting tube is made of silicone.

41. A method of manufacturing a medico-technical device adapted to be engaged with a human or animal body and including a self lubricating element, comprising a step of a plasma treatment in which an element which is not self lubricating is modified such that it becomes the self-lubricating element.
